(19) United States
(12) Patent Application Publication
(10) Pub. No.: US 2006/0210421 A1
(43) Pub. Date: Sep. 21, 2006

(54) Dispenser

(75) Inventors: Geoffrey Robert Hammond, East Yorkshire (GB); Michael Lee, Reading (GB)

Correspondence Address: NORRIS, MCLAUGHLIN & MARCUS, 875 THIRD AVE, 18TH FLOOR, NEW YORK, NY, 10022, US

(73) Assignee: Reckitt Benckiser (UK) Limited, Slough, Berkshire (GB)

(21) Appl. No.: 10/568,463

(22) PCT Filed: Aug. 13, 2004
(86) PCT No.: PCT/GB04/03485

§ 371 Date: May 23, 2006

(30) Foreign Application Priority Data
Aug. 16, 2003 (GB) 03193182

(51) Int. Cl. A61L 9/14

(52) U.S. Cl. 422/3; 422/4; 422/5; 422/123

(57) ABSTRACT

The invention provides an air treatment device comprising: an airborne agent detector comprising a plurality of airborne agent sensors, wherein the airborne agent detector comprises means to detect a threshold level or concentration of an airborne agent; a means to mount a source of air treatment agent to the device; and a means to expel a portion of air treatment agent from a mounted source of agent, upon detection of an airborne agent by the detector.

The present invention relates to a dispenser for air treatment agents, especially for use in deodorising or neutralising odours in an air space.

Air fresheners and other air treatment agents are widely used in many applications, in houses, vehicles and elsewhere. Although they are usually refillable, or cheap and disposable, it is inconvenient to have to fill or replace them often, particularly when many such items are in use for example in a large building. It is also an inconvenience to monitor levels within the devices in order to order refills or new stock as and when the devices become depleted. Furthermore, it can be wasteful to have such devices emitting when not needed.

It would therefore be desirable to extend the life of a substance to be dispensed in an air freshener or odour neutraliser, such as a fragrance for example, in order to reduce costs. One way of extending a life of an air freshener is to include a lid or closure substantially sealing the air freshener to prevent release of the active agents, until a user opens the lid. However, clearly this is inconvenient for the user, and again if a user forgets to re-close the lid after use, unwanted release of the active agents will continue until the device is depleted.

Automated versions of this idea have been proposed, in which a dispensing mechanism turns on and off periodically; set by a user. These systems are adequate when it is possible to predict when dispensing of the active agents is needed; but is inadequate if for example malodour or other substances enter an atmosphere at non-regular intervals.

Efforts have been made to design an air freshener, which dispenses fragrance, deodorant or sanitizing agent only when a room is occupied, and which utilises infrared detectors to detect movement within a room or air space. However, it is rarely necessary to dispense the active ingredient when a person is present in the room, unless said person has undertaken activity, which produced malodour or undesired odours. Thus infrared detection and subsequent release of active ingredient can be relatively wasteful, inefficient and expensive.

The need for efficient non-regular or regular release of air freshener is equally applicable to other active ingredients such as odour neutralisers, anti-bacterial agents, and anti-allergenic compounds; for example if there is a high pollen count within an enclosed space, in order to prevent a person suffering from hay fever from showing symptoms of their predicament. Other allergens include fungal spores, dust mites (and their droppings), pet allergens and the like, for example.

JP 2001 087370 discloses a deodoriser with spraying means for neutralising acid and alkaline odour components when detected by odour sensors.

It would therefore be advantageous to provide an active ingredient release mechanism, which allows portions of air treatment agent to be released from a device only as and when a particular stimulus is present in the air space around the device or within an enclosed space, and which avoids ingredient release as the result of false detection of stimulus.

A problem with devices triggered by very low levels of airborne agent, such as may be detected by the human nose, is that such devices are prone to false detection and triggering, thus wasting the air treatment agent.

Another problem is that such devices may trigger when no humans are present, and so again the air treatment agent may be wasted.

It would also be advantageous to improve the efficiency of release of air treatment agents from devices, into airspaces, in particular maximising the distribution of the agent and enabling release in the optimum efficient manner in response to stimuli in the airspace, or lack of the stimuli. It would also be advantageous to provide an air treatment agent device which is not a line-of-sight device, and which would not be triggered to release an air treatment agent by stimuli other than desired stimuli for said device.

It is the aim of preferred embodiments of the present invention to overcome or mitigate at least one problem of the prior art, whether expressly disclosed herein or not.

According to a first aspect of the invention there is provided an air treatment device comprising: an airborne agent detector comprising a plurality of airborne agent sensors, wherein the airborne agent detector comprises means to detect a threshold level or concentration of an airborne agent; a means to mount a source of air treatment agent to the device; and a means to expel a portion of air treatment agent from a mounted source of agent, upon detection of an airborne agent by the detector.

Preferably the airborne agent detector is of a type whose electrical conductivity is altered, by exposure to the airborne agent.

Preferably the threshold level is at least 0.1 ppm by volume of air of the target airborne agent, more preferably 0.05 ppm, even more preferably 0.01 ppm

Preferably said source is a single source of a target airborne agent.

The device may be of a type in which air treatment agent is expelled only in response to the detection of an airborne agent.

The device may be of a type in which the expulsion of air treatment agent in response to the detection of an airborne agent is not the only way in which air treatment agent is expelled. For example air treatment agent may be passively emanated, and on detection of an airborne agent, an additional portion of air treatment agent is expelled to supplement the background level of passively emanated airborne agent. This may be achieved by various means, for example by expelling a pulse of air treatment agent using a pumping device, or preferably by use of a fan which accelerates the rate of release of the air treatment agent from the passive emanator. In another embodiment, a heater element in proximity to a diffusion wick may be actuated in order to increase the emanation of the air treatment agent.

By airborne agent is meant an airborne chemical in the form of a gas, vapour,solid or liquid particle or droplet.

Suitably the airborne agent detector is operably connected to the means to expel a pulse of air treatment agent, such that the portion of air treatment agent is triggered in response to an airborne agent being detected by the detector.

The means to mount a source of air treatment agent to the device may comprise means to connect a receptacle to the device, the receptacle comprising the air treatment agent. The means to mount a source of air treatment agent may comprise a clip, retaining member, catch, flange, bracket or other similar structure, capable of cooperating with an agent-filled receptacle, and more preferably capable of releasably mounting the agent-filled receptacle.

The portion of air treatment agent may be a pulse of air treatment agent. The portion may be a single pulse. The portion may be a continuous stream of agent over a defined time period, or a plurality of intermittent pulses or streams of agent over a time period which may be pre-determined or be controlled by the device itself and related to the detected level of airborne agent.

The device may be arranged to expel a background level of air treatment agent which may be continuous or intermittent, and the portion of air treatment agent may comprise a booster portion of agent expelled by the device upon detection of an airborne agent by the detector. Thus, for example the device may utilise as an air treatment agent, a deodorant, which may be expelled continuously at a low level to provide constant deodorising action, and upon detection of an airborne agent by the detector, the device may be effected to expel a booster portion of the deodorant to counteract the detected airborne agent. The device may then return to expelling a continuous background level of agent when the detector detects no further airborne agent, or detects an airborne agent under a minimum threshold concentration.

The airborne agent detector may comprise means to detect a single airborne agent or a mixture of airborne agents. The airborne agent detector may comprise means for a user to input which airborne agent or agents the detector is arranged to detect, in use.

The airborne agent detector may comprise means to detect a threshold level of an airborne agent or agents. The expulsion means may only be activated upon detection of the defined threshold, such as a threshold concentration, of an airborne agent, which threshold may be user set or factory set, for example. Thus, only upon detecting said threshold, the detector may operably cooperate with the means to expel a portion of the air treatment agent from the device to activate expulsion of a portion of the air treatment agent.

The expulsion means may continue to expel the portion or a plurality of portions of air treatment agent, until the detector no longer detects an airborne agent or a threshold level of airborne agent.

The dose of air treatment agent expelled is preferably related to the detected level of airborn agent. For instance the dose of air treatment agent released may be proportional to the level of airborne agent detected. For instance the time over which expulsion of airborne agent takes place may be linked to the level of airborne agent detected.

Preferably the airborne agent detector is a gas detector. Thus, preferably the gas detector is arranged to detect a gas and effect expulsion of the portion of air treatment agent from the device in response to detection of the gas.

The gas detector may comprise one or more electronically conductive gas sensors and/or one or more semi-conductive gas sensors.

Preferably the detector comprises one or more semi-conductive sensors.

The gas detector may comprise a plurality of sensors, each sensor comprising a different sensor material. Preferably the gas detector comprises at least 3 sensors, preferably at least 4 sensors, each sensor comprising a different sensor material.

In one embodiment the airborne agent detector is adapted to detect sulphur-containing gases; preferably at least one of hydrogen sulphide, methanethiol (also known as methyl mercaptan) and dimethyl sulphide; more preferably at least two of these; and most preferably all three.

In one embodiment the airborne agent detector is adapted to detect nitrogen-containing gases, preferably at least one of ammonia and nitrogen dioxide, preferably both of these.

In one embodiment the airborne agent detector is adapted to detect carbon monoxide.

The airborne agent detector may be adapted to detect at least two, and preferably all three, of sulfur-containing gases; nitrogen-containing gases; and carbon monoxide.

Useful as semiconductor gas sensors are those gas sensors comprising a metal oxide. Thus preferably the gas detector comprises at least one metal oxide gas sensor, hereinafter referred to as “MOX” gas sensors.

Semi-conducting MOX sensors, heated to approximately 300° C. in air are known to exhibit a strong sensitivity to traces of reactive gases present in the air. The sensitivity is translated into resistance change due to loss or gain of electrons as a result of the target gas reacting with oxygen. The loss or gain of electrons can thus be measured and correlated to determine which gases are present in the air. Thus the loss or gain of electrons can be measured quantitatively as the magnitude of change in electrical resistance, and thus correlates to the concentration of target gas present around the sensor.

Suitable MOX gas sensors include gas sensors comprising oxides of tungsten, tin, any suitable semi conducting metal oxides, such as those comprising zinc, titanium, chromium, cobalt, molybdenum and vanadium, for example.

Particularly preferred MOX gas sensors include sensors comprising one or more of the following metal oxides: SnO₂, WO₃, Cr₂₋ₓTiₓO_{3+z} (where x is from 0.1 to 0.8 and z is determined by the level of vacancies in the material, which is non-stoichiometric: preferably x is from 0.1 to 0.3), TiO₂, ZnO₂, MoO₃ and V₂O₅. The chemical formulae are indicative, as would be known to those in the art, because of the non-stoichiometry of the oxides.

The gas detector may comprise at least one n-type MOX sensor and at least one p-type MOX sensor.

Suitably the MOX sensor comprises a porous film or layer. Since the change in electrical resistance in the sensing electrode is carried by a surface reaction, it is advantageous to maximise the surface area to intensify the response to gas.

Preferably the MOX sensor comprises a metal oxide material connected to a substrate or chip, more preferably an aluminium or silicon substrate or chip. The MOX material is preferably connected to an electrode material, such as platinum or tantalum or a mixture thereof, for example. The electrode material may be inter-digital with the MOX material or may be connected by any other suitable orientation or configuration. There may be an insulating layer on top of the substrate, such as, for example, an oxide layer of the silicon or aluminium substrate between said substrate and said MOX material.

The MOX sensor may also comprise a means for heating the sensor to a required temperature. The means for heating the sensor may comprise a metal member connected to the MOX material and operably connected to a heating means, such as an electrical heating element. The metal member may comprise the same material on the electrode material, where present, and may thus be, for example, platinum or tantalum. The MOX sensor may be heated, in use, to a temperature of at least 300° C.

In particularly preferred embodiments, the MOX sensor comprises a substrate, preferably Si or Al, an oxide layer of the substrate material, a MOX layer comprising inter-digital electrodes, a heating member comprising the electrode material and a temperature sensor.

The MOX sensor may comprise one or more additives to increase the selectivity and/or sensitivity of the MOX material to a particular gas or gases. The additive may be a catalytic additive such as platinum, palladium, gold or titanium, or activated carbon filters, for example. Particularly preferred sensors for detection of sulfur-containing airborne agents are SnO₂ with Platinum and Cr₂₋ₓTiₓO_{3+z}

The MOX sensors may comprise one or more protective coating layers arranged to prevent ablation or damage to the MOX material, in use. The protective coating layer may comprise a membrane, a sintered metal, carbon filter and the like, but the protective coating should not prevent charge transfer on the MOX sensor surface so preferably does not cover the active sensor material.

The gas detector may comprise a conducting polymer (CP) sensor, as an alternative to, or in addition to a MOX sensor.

There are a number of potential advantages in using conducting polymers, over the other sensor technologies, for vapour and gas sensing. There is a far wider choice of materials and hence functional groups with which the gas or vapour can interact, and the materials are often easier to process than inorganic materials, i.e. metal oxides.

Some conducting polymer sensors can operate at room temperature, which is a distinct advantage over the semiconductor sensing technique, as there is a low power requirement. They also show reversible characteristics at room temperature, this means that the recovery rate of the sensors after exposure to target compounds is better than SAW (Surface Acoustic Wave) sensors. The electronic control of the sensor is far less complicated than both semiconductor, MOX and SAW (Surface Acoustic Wave) detection. The CP sensor is stable up to 40° C. and 90% humidity, which is the most significant advantage over the other sensing techniques. Conducting polymer sensors may comprise two gold microelectrodes with an insulating gap between them. The conducting polymer is grown electrochemically across the gap to form a sensor. The conductivity of the polymer is altered by the presence of nucleophilic and electrophilic gases which results in a decrease and increase in the conductivity respectively. Therefore by following the resistance between the two microelectrodes the sensors can be used to sense gases and vapours. The polymers may be doped with anions such as Cl⁻ and SO₄²⁻ which can alter the sensitivity and/or selectivity to different vapours.

Suitable polymers for use in CP sensors include polypyrrole, polyaniline, polythiophene, polypyrorolidone, polyacetylene, polyaraphenylene, polyphthalocyanine, carbon black (or other carbon polymers).

Other sensors that may be used in the gas detector include SAW (surface acoustic wave) sensors, electrochemical cells, optical gas sensors, GASFETS (Gas Field Effect Transistors) pellistors, fibre optic gas sensors, and the like for example.

A gas detector is not a ‘line-of-sight’ detector and is not sensitive to location or orientation. Accordingly the device can be positioned in an out-of-the-way or unobtrusive location without affecting its operation.

In order to prevent a ‘false positive’ detection of gas by a detector, in which a gas similar to that which is arranged to be detected would trigger a release of the air-treatment agent, the gas detector may comprise a plurality of different gas sensors, each of which must preferably detect a specific gas before the air-treatment agent pulse can be released. The plurality of gas sensors may comprise sensors of different materials, each of which may be arranged, to detect the same gas or different gases. Thus, for example the gas detector may comprise an array of metal oxide sensors of different materials, each of which produce a different signal in response to the same gas, and only when a defined combination of signals is emitted by the plurality of detectors will the air treatment agent be released.

Alternatively or additionally some or all of the gas sensors may be arranged to detect different gases and the air treatment agent may only be released when a certain number or concentration of gases is detected.

Alternatively the airborne agent detector may comprise a biosensor or chemical sensor, arranged in use to detect an airborne agent which may be a gas, liquid (including a vapour) or particulate solid.

The biosensor or chemical sensor may be arranged to detect an airborne particle of biological material such as pollen, an allergenic protein, fungal spores, micro organisms, other proteins and the like, for example, or an airborne chemical.

The device may comprise its own power source, such as one or more batteries, for example, or solar cells. Alternatively the device may comprise a plug or socket, arranged in use to cooperate with a corresponding electrical plug or socket, of for example, a mains electricity supply.

Some detectors such as gas sensors, chemical sensors and biosensors generally may have a low power requirement, and therefore a device of the invention using such detectors may be suitable as a portable device utilising an internal power source such as a battery, for example.

The device may include a processor unit which receives the signal(s) produced in response to the airborne agent(s), and determines whether air treatment agent is emitted.

The device may include a person sensor, for example an infra-red sensor (e.g. a PIR sensor). The processor unit may be programmed such that only when a person is present, is the air treatment agent emitted, and only then, in response to the sensing of a target airborne agent.

The processor may be programmed to cause release of air treatment agent only when a sulfur-containing compound is detected.

The processor may be programmed to cause release of air treatment agent only when a nitrogen-containing compound is detected.

The processor may be programmed to cause release of air treatment agent only when carbon monoxide is detected.

The processor may be programmed to cause release of airborne treatment agent when two, or preferably three, of a sulfur-containing compound, a nitrogen-containing compound and carbon monoxide is detected.

The processor may be programmed to cause release of airborne treatment agent only when a sulfur-containing compound is not detected (but when another airborne agent is present).

The processor may be programmed to cause release of airborne treatment agent only when a nitrogen-containing compound is not detected (but when another airborne agent is present, to cause the release of the airborne treatment agent).

The processor may be programmed to cause release of airborne treatment agent only when carbon monoxide is not detected (but when another airborne agent is present, to cause the release of the airborne treatment agent).

The processor may be programmed to cause release of airborne treatment agent only when two of said types of airborne agents are not detected (but when the other type of airborne agent is detected, to cause the release of the airborne treatment agent).

The device may include a timer, such that when the or each detector or sensor detects an airborne agent, air treatment agent is dispensed as a continuous stream for defined period of time, and/or dispensed in a defined number of intermittent pulses. Intermittent pulses may be at regular time intervals or irregular time intervals.

The airborne agent detector, or detectors may be provided with a ASIC (Application Specific Integrated Circuit) circuit as the processor unit, to provide the necessary signals to the air treatment agent dispensing means, in order to activate said dispensing means.

The air treatment agent may be housed in any suitable receptacle, such as a canister, bottle or vial, for example. The receptacle may be a pressurised container such as an aerosol can for example, and may thus comprise, in addition to the air treatment agent, a pressurised gas, preferably a hydrocarbon gas (or hydrocarbon which is a gas at ambient temperature and pressure) such as propane, butane, or pentane, for example, or a halocarbon gas, such as chlorofluorocarbon gases.

The receptacle may be detachably mountable to the device. Thus when the receptacle becomes empty of air treatment agent the receptacle may be removed and either refilled, or another agent filled receptacle mounted on the device.

The air treatment agent expulsion means may comprise any suitable means, such as a pump or aerosol for example, as are known to those skilled in the art. The dispensing means may include a nozzle. The nozzle may comprise an aperture, such as a circular or elliptical hole, or an elongate slot, for example. The nozzle may comprise a plurality of apertures, such as a spray head for example. The plurality of apertures may comprise a mesh.

The expulsion means may simply comprise a wick to enable evaporation of an air treatment agent from the device. Alternatively the expulsion means may comprise ultrasonic expulsion means, nebulising means, electrostatic discharge means and the like, for example.

The nozzle preferably enables the air treatment agent to be dispensed as a spray or fine mist, which may be effected by forcing the agent through a plurality of restricted size apertures, or the like, for example.

The air treatment agent preferably comprises an agent capable of masking, neutralising or retarding malodour, or unwanted odour in an airspace around the device. The air treatment agent may comprise a deodorant, an anti-bacterial agent, a sanitizing agent, a fragrance or a perfume, for example. The air treatment agent may comprise an anti-allergenic material, preferably arranged to react with and/or neutralise an allergen detected by the airborne agent detector, in use.

The air treatment agent may comprise a solid in the form of granules or powder, but preferably comprises a liquid or gas, at ambient temperature and pressure. Preferably the air treatment agent comprises a liquid, which may be dispensed in the form of a fine spray or mist through a suitable nozzle. If the air treatment comprises a gas or liquid, it may comprise a gas or vapour capable of reacting with the airborne agent to be detected in order to neutralise any malodour associated with the airborne agent.

By gas detector we mean a detector capable of detecting a gas or vapour per se, and/or fine particulate solids or liquid droplets dispersed in gases or air.

The device may comprise a fan or similar means, operably connected to the air treatment agent dispensing means. The fan may comprise part of the means to expel a portion of air treatment. The fan is preferably arranged to activate immediately prior to and/or during activation of the dispensing means, in order to effect increased speed of expulsion of the air treatment agent from the device, and/or to increase the distribution of the agent in the airspace surrounding the device.

The fan is preferably operably connected to the airborne agent detector, such that, upon detection of the airborne agent by the detector, the fan is activated prior to or during activation of the expulsion means.

The device may comprise a heater, operably connected to the air treatment agent dispersing means. The heater may be arranged to activate immediately prior to and/or during activation of the air treatment agent expulsion means, in order to effect heating of the portion of air treatment agent as it is expelled from the device. Thus the heater may be used to vaporise, or render more fluid, a portion of air treatment agent expelled from the device.

The heater may be arranged to heat the portion when said portion is within the device or agent receptacle; alternatively the heater may be arranged to heat the portion as it leaves the device. The heat may also serve to improve distribution of the air treatment agent through convection and may activate the air treatment agent molecules, if the air treatment agent comprises a composition which can be activated by heat, or which effects increased efficacy on heating.

The device may include an alarm, operable when a gas is sensed which is dangerous. For example the device may have an alarm triggered by a threshold level of carbon monoxide.

According to a second aspect of the invention there is provided a device of the first aspect of the invention on which is mounted a source of air treatment agent.

According to a third aspect of the invention there is provided a method of treating an airspace with an air treatment agent, the method comprising the steps of detecting an airborne agent in an airspace and activating expulsion of an air treatment agent into the airspace in response to detection of the airborne agent.

The method may comprise providing an airborne agent detector, a source of air treatment agent and a means to expel a portion of air treatment agent means upon detection of an airborne agent by the detector.

The method may comprise expelling a single portion of agent in response to detection of an airborne agent, or may comprise dispensing a plurality of portions intermittently, whether at regular or irregular intervals. Alternatively the expulsion of agent may comprise expelling a continuous stream of agent for a defined time period upon detection of gas. The expulsion means may expel a continuous portion or intermittent portions of agent for as long as the detector detects an airborne or a defined threshold level of an airborne agent, or for a shorter or longer period of time, for example.

The portion(s) may be dispensed as a pulse of agent from the dispensing means.

For example, in the case of the detector detecting a gas produced by tobacco smoking, or a mixture of gases, the expulsion means may be effected to expel a single portion of air treatment agent, or may be effected to expel a plurality of portions for a defined time period or for such a time as the detector continues to detect the gas or gases. In some embodiments the expulsion means may also be arranged to expel one or more portions of agent when the gas detector signals that no more further gas has been detected.

Alternatively, the expulsion means may dispense the portion continuously over a defined period of time, which period of time may be predefined by a user, or may correspond to a time period shorter than, equal to or longer than the time period during which the airborne agent detector detects an airborne agent or defined threshold level of an airborne agent.

Preferably the method comprises treating an airspace within a room, whether domestically (such as a kitchen, living room, bathroom, bedroom, toilet, garage, basement, loft, etc) commercially, or industrially. The method may comprise treating an airspace within an object, whether a closed object or an open object. Suitable objects include dishwashers, washing machines, dustbins and other waste receptacles, wardrobes, laundry baskets, bags, shoes, vehicle interiors, refrigerators, cupboards, toilets, sanitary bins, nappy containers, sharps bins, and the like for example.

The airborne agent detector, air treatment agent expulsion means, and source of air treatment agent may be as described for the first aspect of the invention.

According to a fourth aspect of the present invention there is provided the method of the third aspect using the device of the first or second aspect.

For better understanding of the invention and to exemplify how embodiments of the same may be put into effect, the invention will now be described by way of example with reference to the accompanying drawings in which:

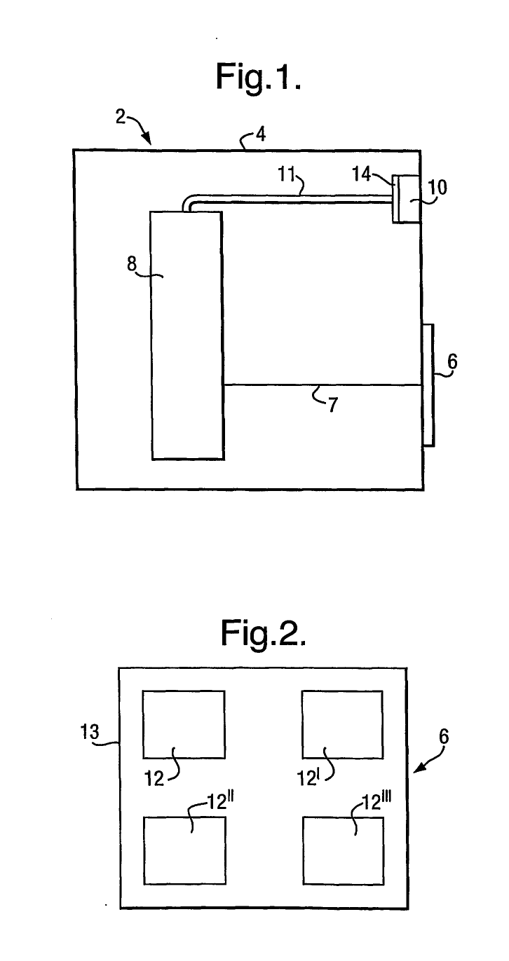

FIG. 1 illustrates a schematic view of a dispenser in accordance with the invention;

FIG. 2 illustrates a plan view of the MOX sensor of the device shown in FIG. 1;

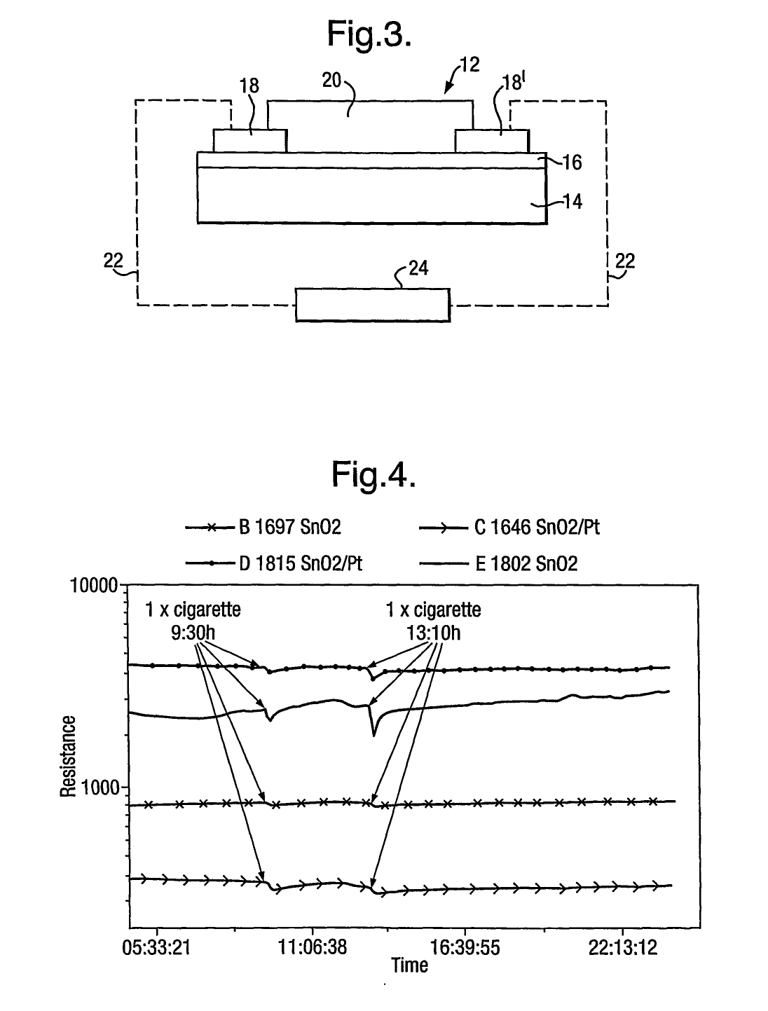

FIG. 3 illustrates a side sectional view of one of the MOX sensors of the MOX sensor array shown in FIG. 2;

FIG. 4 shows the results of an experiment using the device of FIGS. 1 to 3, including MOX gas sensors, in simulated domestic conditions to sense gases produced by tobacco smoking;

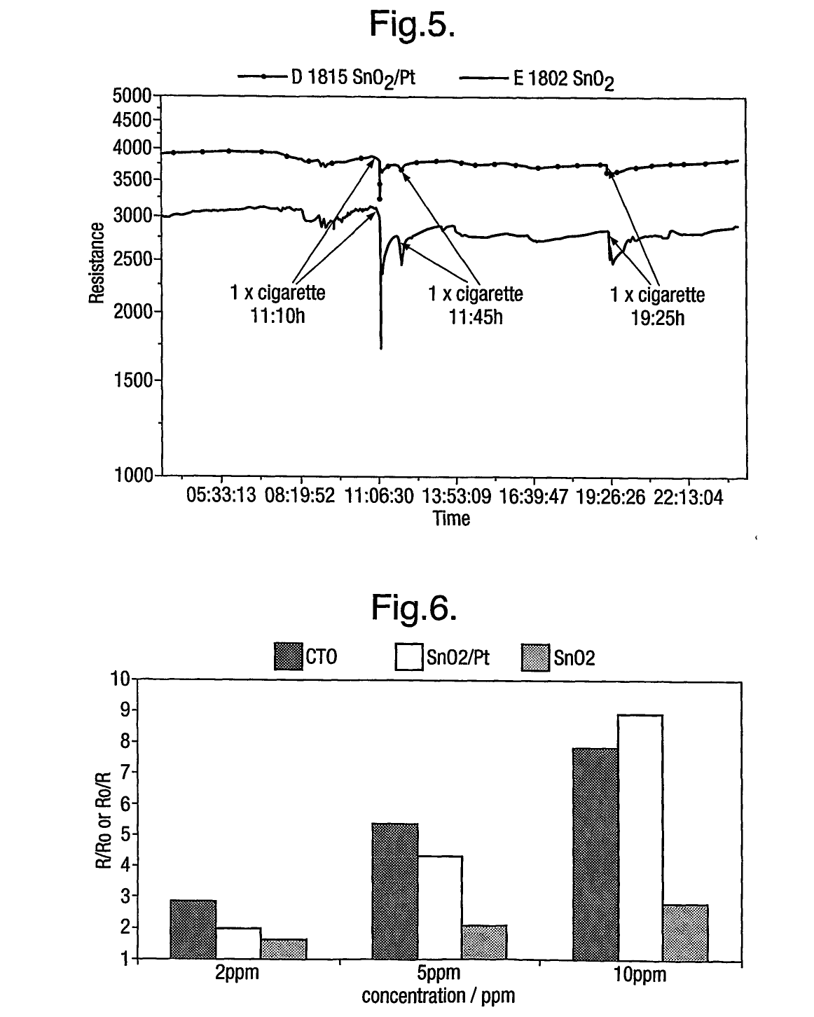

FIG. 5 shows the results of a second experiment using the device of FIGS. 1 to 3, in simulated domestic conditions; and

FIGS. 6 to 8 show the results of further experiments, with sulfur-containing gases.

We refer firstly to FIG. 1 which illustrates a side sectional schematic view of an air treatment dispensing device 2 the invention.

The device comprises a housing 4 on which is located an airborne agent detector in the form of a gas detector, comprising a gas sensor array 6. Within the housing 4 is located a source of air treatment agent in the form a detachable canister 8 which comprises a liquid deodorant as an air treatment agent. The canister 8 is in electronic communication with the sensor array 6 via an electrical circuit 7. The canister 8 comprises an outlet conduit 11, at the end of which opens to a nozzle 10 which comprises a plurality of apertures (not shown) which enable deodorant to exit the housing 4 as a fine spray or mist, when the device 2 is used. Situated within the nozzle 10 is a fan 14, through which the outlet conduit 11 extends. The fan 14 is arranged in use to be actuated upon expulsion of a portion of deodorant from the outlet conduit 12 into the nozzle 10, in order to that the expelled portion is forced through the apertures of the nozzle 10, in order to increase distribution of the fine spray of mist outside of the device 2.

We turn now to FIGS. 2 and 3, which illustrate a front view and side sectional view of the sensor array 6 of FIG. 1. The sensor array 6 comprises a substrate 13 comprising a silicon base 14 as shown in FIG. 3 on which is laid an insulating SiO₂ layer 16 as shown in FIG. 3. On top of the SiO₂ layer are positioned four metal oxide (MOX) sensors 12, 12′, 12″, 12′″. The four MOX sensors 12, 12′, 12″, 12′″ comprise materials 20: SnO₂, SnO₂/Pt, SnO₂ and SnO₂/Pt respectively.

Each MOX sensor 12, 12′, 12″, 12′″ further comprises its own abutting underlayer portion of the silicon substrate 14 and SiO₂ layer 16, and two spaced apart platinum electrodes 18, 18′, the span of which is bridged by the MOX sensor material 20. The electrodes are connected to a voltmeter 24 which can determine resistance across the sensor material of the sensors 12, 12′, 12″ and 12′″, via electrical wires 22.

Each of the MOX sensors 12, 12′, 12″, 12′″, is operably connected to a heating member in the form of a Ta/Pt resistance layer connected to the sensor material 20 of the four sensor array 6 and which contacts each of the four MOX sensors.

Use of the device 2, will now be described with reference to FIGS. 1 to 3 and FIGS. 4 and 5.

It is known that semi-conducting MOX sensors heated to approximately 300° C. in air, exhibit strong sensitivity to traces of reactive gases present in the air. The measurement effect is commercially exploited for only a relatively few number of oxides due to the requirement for a unique combination of resistivity, magnitude of resistance change in a specific gas (sensitivity) and humidity effects. Amongst the oxides which are used as MOX sensors are SnO₂, as used in the sensor array 6 of the device 2 described hereinabove. The SnO₂ sensors can be enhanced, selectivity wise and sensitivity wise by the use of catalytic additives, such as the Pt present in sensors 12′ and 12′″ of the device 6.

The resistance change induced by the sensors is caused by loss or gain of the surface electrons as a result of absorbed oxygen reacting with a target gas. If the oxide is an n-type, there is either a donation (producing gas) or subtraction (oxidizing gas) of electrons from the conduction band within the material. The result is that n-type oxides increase their resistance when oxidizing gases such as NO₂, O₃ are present while reducing gases such as CO, CH₄, and ethanol lead to a reduction in the resistance. The converse is true for p-type oxides, where electron exchange due to gas interaction leads either to a rise (oxidizing gas) or a reduction (reducing gas) in electron holes in the valence band. Each of these reactions then translates into corresponding changes in electrical resistance. Unlike some of the gas sensing technologies, MOX sensors can be made quantative, as the magnitude of change in electrical resistance is a direct measure of the concentration of the target gas present.

The sensors 12, 12′, 12″, 12′″, were selected due to their advantageous properties in detecting NO₂, O₃, CO, CH₄ and ethanol, as are commonly produced as gases through smoking tobacco. Thus the device 6 which utilizes the sensor materials given above is particularly suited to sensing gases produced in tobacco smoking in a confined or semi-confined airspace.

Since the change in electrical resistance in the sensing oxide of sensors 12, 12′, 12″ and 12′″ is caused by surface reaction, it is advantageous to maximize the surface area to intensify the response to the gas. For this reason, the sensors 12, 12′, 12″ and 12′″ include a layer of MOX material 20 which is in the form of a thin film. Alternatively the layer 20 may be slightly thicker, but highly porous. The MOX material 20 is either printed down or deposited onto the semi-conductive layer 16. The electrodes 18, 18′ are coplanar and located at the MOX material 20/semiconductor layer 16 interface. In the sensor array 6 shown in FIG. 2, the SiO₂ insulating layer 16 is approximately 1 μm thick. The Ta/Pt inter-digital electrodes 18, 18′ are approximately 200 nm thick but may be anywhere between 10 nm and 1000 nm thick.

Selectivity can be enhanced further if desired through the use of different metal oxide layers 20 in each of the sensors, or use of catalytic additives, different operation temperatures, protective coatings and activated carbon filters, for example.

Upon detection by the sensors 12, 12′, 12″ and 12′″, and upon lowering of the resistance as shown in FIG. 4, the sensor array 6 emits a signal via electrical circuit 7 to the canister 8 to effect dispensing of a portion of the deodorizing agent within the canister. Upon receipt of the signal, a pump (not shown) within the canister 8 actuates to pump a portion of the deodorizing agent through the outlet conduit 11 and through the nozzle 10 of the device 2. As the canister 8 pumps out the portion of a treatment agent, the fan 14 is actuated. Thus as the agent enters the nozzle 10, the fan effects increased dispersion of the agent from the nozzle 10 through the apertures (not shown), such that the spray or mist of the treatment agent reaches further into the airspace in which the device 2 is situated.

In use the air treatment device 2 is located within an airspace to be treated, such as a room, refrigerator, sanitary bin, sharps bin or the like etc.

Use of the device 2 will now be described by way of an experimental example. The device 2 was utilized in a living a room of a two person household, where tobacco smoking took place.

The device 2 was mounted to a wall within the living room of a household in Hessle, UK, and activated to detect a combination of gases produced in combustion of tobacco through persons in the room smoking cigarettes.

In particular, the sensor material 20 of the sensors 12, 12′, 12″, 12′″ of the device 6 are able to detect NO₂, O₃, CO, CH₄ and ethanol, which are common gases produced through combustion of tobacco.

The device 6 was activated, and a person entered the room at a predetermined time 9.30 am, and lit a cigarette. Approximately 2½ hours later a second cigarette was lit within the room by the same person. FIG. 4 shows the output results of the four sensors 12, 12′, 12″, and 12′″, in response to detection of gases produced by the cigarette smoke within the airspace. As can be seen from FIG. 4, as the first cigarette was lit at 9.30 am, the sensors 12, 12′, 12″ and 12′″ recorded a decreasing resistance across the sensor material 20. When the second cigarette was lit at 1.10 pm, again the four sensors 12, 12′, 12″ and 12′″ recorded a decrease in resistance across the sensing material 20.

Upon detection by the sensors 12, 12′, 12″ and 12′″, and upon lowering of the resistance as shown in FIG. 4, the sensor array 6 emitted a signal via electrical circuit 7 to the canister 8 to effect dispensing of a portion of the deodorizing agent within the canister. Upon receipt of the signal, a pump (not shown) within the canister 8 actuated to pump a portion of the deodorizing agent through the outlet conduit 11 and through the nozzle 10 of the device 2. As the canister 8 pumped out the portion of a treatment agent, the fan 14 was actuated. Thus as the agent entered the nozzle 10, the fan effected increased dispersion of the agent from the nozzle 10 through the apertures (not shown), such that the spray or mist of the treatment agent reached further into the living room in which the device 2 was situated.

FIG. 5 shows the results of a second experiment in which the device 6 was placed in a second living room at a household in Freiburg, Germany. Three cigarettes were smoked during the day at 11.10 am, 11.45 am and 7.25 pm. The device 2, for this experiment, was utilised with only two sensors, 12 and 12′, corresponding to the SnO₂/Pt and SnO₂ materials as sensor material 20. It can be seen that immediately upon lighting a cigarette at 11.10 am, 11.45 am and 7.25 pm resistance was lowered across the MOX material 20 of the sensors 12 and 12′, which induced a signal, which was subsequently emitted via the control circuit 7 to the canister 8. The canister 8 then actuated release of a portion of deodorizing air treatment agent out of the device 2 via the nozzle 10 as described herein before, in order to mask the tobacco gas malodour.

Thus the device 2 can be used effectively to counter malodour produced by tobacco smoking or other malodour produced within a confined airspace. Sensor 2 may be situated in any confined or semi-confined airspace where malodours occur. The sensor material 20 may be changed to increase selectivity and/or sensitivity to varying gases which may be produced as part of a malodour.

In alternative embodiments, instead of MOX sensor material, conducting polymer (CP) sensors may be utilised. There are a number of potential advantages in using conducting polymers, over the other sensor technologies, for vapour sensing. There is a far wider choice of materials and hence functional groups with which the vapour can interact, and the materials are often easier to process than inorganic materials, i.e. metal oxides. Some conducting polymer sensors can operate at room temperature, which is a distinct advantage over the semiconductor MOX sensing technique, as there is an inherent low power requirement. They also show reversible characteristics at room temperature, this means that the recovery rate of the sensors after exposure to target compounds is better than SAW (Surface Acoustic Wave) sensors. The electronic control of the sensor is far less complicated than both semiconductor MOX and SAW detection. The CP sensor is stable up to 40° C. and 90% humidity, which is the most significant advantage over the sensing techniques.

The conducting polymer sensors are essentially two gold microelectrodes with an insulating gap between them. The conducting polymer is grown electrochemically across the gap to form the sensor. The conductivity of the polymer is altered by the presence of nucleophilic and electrophilic gases which results in a decrease and increase in conductivity respectively. Therefore by following the resistance between the two microelectrodes the sensors can be used to sense gases and vapours. The polymers may be doped with anions such as Cl⁻ and So₄²⁻, which can alter the sensitivity to different vapours.

The conducting polymer, once coated onto the electrode material, requires activation before use as a chemical sensor. Activation is required to convert the insulating, neutral form of the polymer to oxidized, positively charged, conducting form where anions from an electrolyte solution are incorporated into the polymer film. To achieve this the polymer films are first characterized in a base electrolyte by another electrochemical process called cyclic voltammetry. Here the potential is cycled between certain limits at a chosen scan rate for at least two complete cycles. The point at which an oxidation peak occurs gives the maximum potential required for activation, and potentials above this which cause over oxidation and degradation of the conducting polymer film.

Other gas detectors that may be used alternatively or additionally to MOX and CP based gas detectors include those comprising Surface Acoustic Wave sensors and/or sensor materials.

In further embodiments the portion of dispensing agent dispensed upon detection of a gas or plurality of gases by the sensor array 6 may comprise a plurality of intermittent pulses, whether at regular or irregular time intervals, or may comprise a continuous dispersal of a stream of air treatment agent over a defined period of time. The defined period of time may be user defined or preset in the device 2. The device 2 may emit a constant background level of air treatment agent and expel a portion, in the form of a booster portion upon detecting an airborne agent in an airspace.

The device 2 may include a heater, in other embodiments, in addition to or alternative to the fan 14. The heater may be arranged to render any air treatment agent expelled through the nozzle 10 more fluid or vaporize a liquid air treatment agent. The heater may even activate air treatment agents which comprise heat-activated compounds. Other air treatment agent expulsion means may include nebulisers, electrostatic means, a simple wick or the like for example.

In yet further alternative embodiments the portion of air treatment agent to be dispensed may be effected to be dispensed immediately upon detection of a gas, or at any defined time interval after detection of a gas. The fan 14 may be effected to continue operation after the portion of air treatment agent has been dispensed, in order to further encourage the air treatment agent to disperse around the airspace after the device 2 has been activated.

The device 2, may comprise, instead of a gas detector, a detector in the form of a biosensor or chemical sensor. The biosensor or chemical sensor may be arranged to detect a particulate solid, liquid or gas in air, and may be arranged to detect chemical agents or biological material such as proteins, microorganisms, allergens, fungal spores and the like for example. The biosensor or chemical sensor may be any suitable sensor such as an amperometric sensor, optical sensor, or the like, for example, as are well known to those skilled in the art.

Further experiments were carried out with a device comprising four sensors, namely: SnO₂; [SnO₂+Pt] (in series); CTO (Cr_{1.8}Ti_{0.2}O_{3+z}); and WO₃. All were set on a common silicon wafer, on a common quartz substrate.

The target gases were H₂S, (CH₃)₂S and CH₄S. The tests were carried out under ambient conditions, with the usual heating of the sensors.

FIG. 6 shows the change in resistance across the CTO, SnO₂ and [SnO₂+Pt] sensors (R=resistance; Ro=original resistance) at concentrations of 2 ppm, 5 ppm and 10 ppm of (CH₃)₂S. The CTO and [SnO₂+Pt] sensors appear to be particularly discriminating.

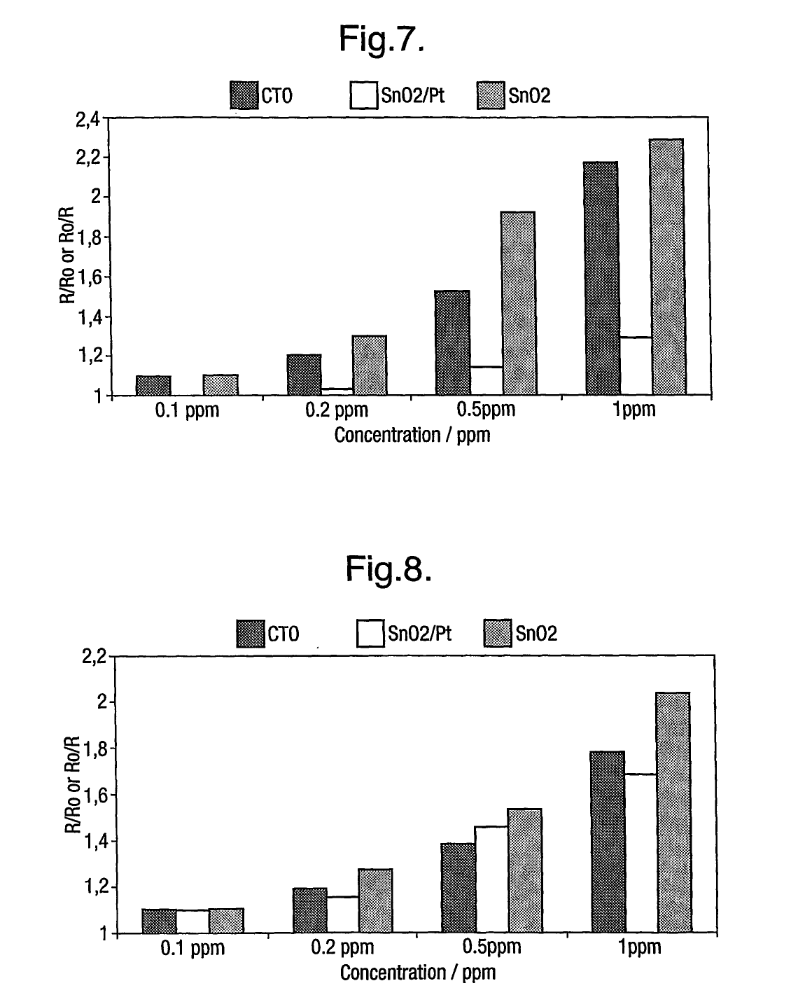

FIG. 7 shows corresponding results for CH₄S at concentrations of 0.1 ppm, 0.2 ppm, 0.5 ppm and 1 ppm by volume. In the case of this gas the CTO and SnO₂ sensors appear particularly discriminating.

FIG. 8 shows corresponding results for H₂S at concentrations of 0.1 ppm, 0.2 ppm, 0.5 ppm and 1 ppm by volume. In the case of this gas, all the sensors tested appeared to be discriminating.

Tests were also carried out on the unit for which results are given in FIGS. 6 to 8, but using CO, NO₂ and NH₃, in turn, as the gas. These are regarded in this example only as interfering or rogue gases in the context of detecting the target gases; it is not wished that they trigger release of airborne agent in this example. It was found that they gave a small change in sensor resistance at normal levels; such that they would not release of airborne agent. If necessary a sensor “tuned” to CO, NO₂ or NH₃ could be provided, such that if that sensor fired, the device either would not trigger the release of airborne agent, or would only do so if an especially high level of H₂S, CH₄s or (CH₃)₂S was detected.

1. An air treatment device comprising: a gas or vapour detector comprising a plurality of gas or vapour sensors, wherein the gas or vapour detector comprises means to detect a threshold level or concentration of a gas or vapour; a means to mount a source of air treatment agents to the device; and a means to expel a portion of air treatment agent, upon detection of a gas or vapour by the detector.

2. An air treatment device according to claim 1 wherein the mounted source of air treatment agent also passively emanates the air treatment agent.

3. An air treatment device according to claim 1 wherein the means to expel a portion of air treatment agent comprises a heater element in proximity to a diffusion wick, the heater element being actuated upon detection of a gas or vapour by the detector in order to increase the emanation of the air treatment agent.

4. An air treatment device according to claim 1, wherein the means to mount a source of air treatment agent to the device comprises means to connect a receptacle (8) to the device, the receptacle (8) comprising the air treatment agent.

5. An air treatment device according to claim 1, wherein the device includes a processor unit to determine when the signals from the gas or vapour sensors (6) cause expulsion of a portion of air treatment agent.

6. An air treatment device according to claim 1, wherein the device comprises at least two sensors which sense the same gas or vapour and the processor unit receives signals from both sensors in order to cause a portion of airborne treatment agent to be expelled.

7. An air treatment device according to claim 5, wherein the detector includes a sensor which detects both a target gas or vapour and a non-target gas or vapour, wherein in order to eliminate expulsion of air treatment agent in response to the non-target gas or vapour, the device includes a second sensor which detects the non-target gas or vapour but not the target gas or vapour, the processor unit being arranged to prevent expulsion of the air treatment agent when the second sensor detects a signal, completely or until the first sensor gives a signal at a higher threshold value than usual.

8. An air treatment device according to claim 5, wherein the detector includes a person detector, and the processor unit allows airborne treatment agent to be expelled, in response to a signal from one or more of the sensors, only when the person detector gives a signal and for an interval thereafter.

9. An air treatment device according to claim 1, wherein the detector comprises a conducting polymer sensor.

10. An air treatment device according to claim 1 comprising at least one metal oxide sensor.

11. An air treatment device according to claim 1, wherein the air treatment agent expulsion means comprises a pump or aerosol.

12. An air treatment device according to claim 1, on which is mounted a source of air treatment agent.

13. An air treatment device according to claim 1, wherein the air treatment agent comprises an agent capable of masking, neutralising or retarding malodour, or unwanted odour.

14. An air treatment device according to claim 1, wherein the air treatment agent comprises a deodorant, an anti-bacterial agent, a sanitizing agent, a fragrance, a perfume or an anti-allergenic agent.

15. A method of treating an airspace with an air treatment agent, the method comprising the steps of: providing an air treatment device according to claim 1, and detecting a gas or vapour in an airspace and activating expulsion of an air treatment agent into the airspace in response to detection of the gas or vapour, using an air treatment device.

16. The method according to claim 15, comprising the further step of expelling a single portion of agent in response to detection of an airborne agent, or a plurality of portions intermittently.

17. The method according to claim 15, wherein expulsion of an agent comprises expelling a continuous stream of agent for a defined period of time upon detection of an airborne agent.
